(19) United States
(12) Patent Application Publication
(10) Pub. No.: US 2018/0243061 A1
(43) Pub. Date: Aug. 30, 2018

(54) METHOD FOR TREATMENT PLANNING

(71) Applicant: SICAT GMBH & CO. KG, BONN (DE)

(72) Inventors: TOBIAS LEHNER, BONN (DE); JOCHEN KUSCH, WACHTBERG (DE); NILS HANSSEN, BONN (DE)

(73) Assignee: SICAT GMBH & CO. KG, BONN (DE)

(21) Appl. No.: 15/757,653

(22) PCT Filed: Sep. 6, 2016
(86) PCT No.: PCT/EP2016/070965

§ 371 Date: Mar. 6, 2018

(30) Foreign Application Priority Data
Sep. 8, 2015 (DE) 10 2015 115 034.4

(51) Int. Cl. A61C 19/045; A61C 9/00; A61B 5/00; A61C 13/34

(52) U.S. Cl. CPC A61C 19/045; A61C 9/0006; A61C 7/002; A61C 13/34; A61C 9/004; A61B 5/4547

(57) ABSTRACT

A method for planning a treatment includes recording a first data record of three-dimensional image data of surfaces of an upper and lower jaw. A bite plate with cured impressions of the upper and lower jaw and markers is fitted to the patient. A second data record is recorded of three-dimensional image data of surface data of the bite plate without the patient. A movement recorder is fastened to the lower jaw while the patient bites on the bite plate. The bite plate is removed from the patient. Movements of the movement recorder during mastication movements are recorded and stored in a third data record as movement data. The three-dimensional image data of the first data record is combined with the movement data. A registration is performed using the second data record. The mastication movements of the lower jaw relative to the upper jaw are displayed for treatment planning.

### CROSS REFERENCE TO PRIOR APPLICATIONS

This application is a U.S. National Phase application under 35 U.S.C. § 371 of International Application No. PCT/EP2016/070965 filed on Sep. 6, 2016 and which claims benefit to German Patent Application No. 10 2015 115 034.4, filed on Sep. 8, 2015. The International Application was published in German on Mar. 16, 2017 as WO 2017/042156 A1 under PCT Article 21(2).

### FIELD

The present invention relates to a method and to a system for planning a treatment on the set of teeth of a patient, wherein the planning is based on a representation of a mastication movement of the patient on a computer screen, the upper and the lower row of teeth moving in relation to one another during the mastication movement.

### BACKGROUND

Imaging methods and electronic registrations are included among the most important methods for capturing the functional state of the stomatognathic system and for planning complex prosthetics restorations. Various methods for visualizing anatomical relationships in the living body are known in order to simplify the diagnosis for a treating medical practitioner and to facilitate an optimized therapy planning. DE 10 2012 104 912 A1, for example, describes the anatomical and in particular also the functional kinematic representation of jaw joints in volumetric and surface views in three dimensions. It is here possible to present a digital volumetric lower jaw image in different positions in relation to a digital volumetric upper jaw image. Condylography is used to record a movement and a multiplicity of position data records are stored (“condylogram”). Such a position data record describes the real spatial bearing of the lower jaw in relation to the upper jaw at a certain point of the movement. In the method, the data records are computationally “simulated” on the basis of a first data record that is recorded by a volume tomographic method, taking account of the ascertained movement data contained in the condylogram, and are presented to the observer on the screen.

Such a method has also been described in the article “SICAT Function: Anatomical Real-Dynamic Articulation by Merging Cone Beam Computed Tomography and Jaw Motion Tracking Data”, International Journal of Computerized Dentistry 2014, 17(1); 65-74. A bite plate (“FusionBite”), which has dental impressions of the patient, and consequently impressions of the two rows of teeth in a cured compound plays a central role in the described procedure. The use of this bite plate makes it possible to have an exact spatial assignment between 3D x-ray data and the recorded (mastication) movement data. The bite plate is worn by the patient during an x-ray recording. After the data is assigned in a spatially exact manner with the aid of the bite plate, the movement of the jaw joints can be reproduced with a computer program. The simulated movement of the digital tooth impressions can in turn be presented on a screen and can be examined exactly on the basis of the moving representation.

The volume tomographic data in the known methods are recorded by an x-ray scan on the patient who is subjected to corresponding x-ray exposure during the examination. The recording region in many x-ray devices is too small to be able to see the jaw joints in the recording. The recording region of such small x-ray devices suffices, however, for these to be used for the above-described procedure and for the patient-individual movement of the rows of teeth to be simulated on the basis of the digital tooth impressions. The patient in such x-ray devices is also exposed to ionizing radiation in order to establish the spatial assignment via the bite plate.

DE 10 2013 204 207 A1 describes a bite fork via which it is possible to establish a relationship between the measurement data of an intraoral 3D surface scanner and the data of a 3D positioning system and capture the dental arch in order to be able to represent the surfaces statically and in motion.

DE 10 2010 021 934 A1 describes a similar dental tool for obtaining correlation data, the tool interacting with a sensor system held on a frontal arch in order to be able to carry out movement measurements.

### SUMMARY

An aspect of the present invention is to develop a method, implementable by simple devices, for representing the mastication movement, the method minimizing radiation exposure of the patient, allowing for anatomical peculiarities to be easily determined, and allowing the planning of prosthetic restorations to be undertaken while taking into account individual mastication habits of a patient.

In an embodiment, the present invention provides a method for planning a treatment on a set of teeth of a patient. The planning is based on a representation of mastication movements of the patient in which an upper row of teeth and a lower row of teeth move in relation to one another. The method includes recording a first data record with a camera system. The first data record comprises three-dimensional image data of surfaces of an upper jaw which is at least partly toothed and surfaces of a lower jaw which is at least partly toothed. A bite plate is fitted to the patient. The bite plate comprises cured impressions of the upper jaw, cured impressions of the lower jaw, and identifiable markers arranged at least one of at and in the bite plate. A second data record is recorded comprising three-dimensional image data which comprises surface data of the bite plate without an involvement of the patient. The second data record allows for an identification of the surfaces of the upper jaw, the surfaces of the lower jaw, and the identifiable markers of the bite plate therein. A movement recorder is detachably fastened to the lower jaw of the patient while the patient bites on the bite plate fitted on the patient so as to register an orientation of the movement recorder in relation to the bite plate. The bite plate is removed from the patient. Movements of the movement recorder during the mastication movements are recorded using a detector detachably fastened to a head of the patient. The movements are stored in a third data record as movement data. The three-dimensional image data of the first data record is combined with the movement data of the third data record. A registration is performed using the second data record. The mastication movements of the lower jaw in relation to the upper jaw are presented on a screen for the purposes of planning the treatment.

### BRIEF DESCRIPTION OF THE DRAWINGS

The present invention is described in greater detail below on the basis of embodiments and of the drawings in which:

FIG. 1 shows a bite plate which has not yet been equipped with an impression compound;

FIG. 2 shows a bite plate which has not yet been equipped with an impression compound surrounded by a cylinder made of a thin sheet which partly absorbs x-ray radiation during an x-ray scan;

FIG. 3 shows the system for recording movement data detachably fastened to the lower jaw of a patient via a holder;

FIG. 4 shows a detailed view of the system for recording movement data;

FIG. 5 shows a simulation of the mastication movement using image data, namely the rows of teeth of upper jaw and lower jaw presented on a screen with the set of teeth being completely closed;

FIG. 6 shows a simulation of the mastication movement using image data, namely the rows of teeth of upper jaw and lower jaw presented on a screen with the set of teeth being opened to a maximum extent; and

FIG. 7 shows a simulation of the mastication movement using image data, namely the rows of teeth of upper jaw and lower jaw presented on a screen as a frontal view of the open set of teeth.

### DETAILED DESCRIPTION

The essence of the present invention lies in completely dispensing with the x-ray scan on the patient and carrying out the x-ray scan, which is ultimately necessary for registering the data records, and consequently for the exact fusioning thereof, on a model representing the bite of the patient. According to the present invention, a known type of bite plate is fitted to the patient for this purpose, the bite plate having cured impressions of the at least partly toothed upper jaw and of the at least partly toothed lower jaw. The bite plate is provided with identifiable markers in order to be able to track the orientation of the bite plate in the second data record of the scanned data in an improved manner. The second data record containing three-dimensional image data of the bite plate is then recorded without patient involvement. This second data record should be recorded so that the surfaces of the toothed jaws and the markers of the bite plate can be identified.

Two options exist for recording the second data record according to the present invention. It is firstly possible to record only the bite plate with the cured impression compound situated thereon, into which the tooth impressions have been dug. The “positive” surfaces of the toothed jaws can then be calculated retrospectively from the “negative” tooth impressions. If only the bite plate is scanned using a 3D x-ray method, it is advantageous to use a radiopaque impression compound in order to be able to identify the (negative) contour of the teeth in an improved manner.

In an embodiment of the present invention, the bite plate can, for example, have a plug-in adapter (“flange”) in the front region via which a rigid mechanical connection to the recording system for creating a second data record, in particular to the x-ray device, is possible.

It is secondly possible to make the second data record from a model of the set of teeth, in particular from a plaster cast model, with an upper and lower jaw of the patient. The surfaces of the toothed jaws are available as “positive” surfaces in the model. The plaster cast models lie in the impressions of the bite plate in an interlocking manner during the scan.

The scan to be made of the bite plate is undertaken using a method allowing the negative and/or positive surfaces to be resolved. In certain circumstances, it is possible to record a three-dimensional data record using an optical camera scan. It is advantageous, however, if the scan is recorded using the x-ray radiation from a digital volume tomography (DVT) scanner. The use of the digital volume tomography (DVT) scanner makes it possible to exactly represent the surfaces of the bite plate and/or of the model. If use is made of x-ray radiation, it is advantageous to make the markers on the bite plate from a radiopaque material, for example, in the form of small, introduced spheres, so that they can be identified well in the three-dimensional data record.

In detail, the method for planning a treatment on the set of teeth of a patient appears in the following steps on the basis of the representation of a mastication movement of the upper and lower row of teeth of the patient:

Firstly, an intraoral camera system is used to record a first data record with three-dimensional image data of the surfaces of the upper jaw that is toothed to a greater or lesser extent and of the lower jaw that is toothed to a greater or lesser extent. The jaws can be visualized on a screen on the basis of the data both during and after the recording. The method also works with partly toothed jaws. There only need to be enough teeth present to allow a registration to be carried out.

In the meantime, a bite plate (“FusionBite”) is fitted to the patient via the patient biting into the impression compound applied onto the bite plate. After curing, the bite plate has the cured impressions of the toothed upper jaw and of the toothed lower jaw. In order to be visible in the subsequent scan, identifiable markers with a defined form are provided at and/or in the bite plate, the markers consisting of a radiopaque material in order to be visible in the x-ray scan. The markers have a known spatial relationship to the plug-in adapter of the FusionBite in order to facilitate a spatially exact registration with the movement data.

According to the present invention, the second data record with three-dimensional image data containing surface data of the bite plate and, optionally, of a model is then recorded without any patient involvement. The recording technique should be selected so that the surfaces of the toothed jaws and the markers of the bite plate can be identified in the data.

For the purposes of recording the three-dimensional image data with surface data of the bite plate and of the model, it is advantageous if the bite plate is surrounded by a metal sheet, in particular a cylindrical metal sheet, for example, a cylindrical metal sheet made of copper or aluminum, with a varying thickness. The metal sheet can be connected to the bite plate via the adapter. The metal sheet brings about an x-ray attenuation similar to that of the soft tissue of the (missing) patient. The x-ray recordings therefore have similar grayscale value characteristics to a real patient scan.

A movement recorder is fastened in a detachable manner to the lower jaw of the patient to record movement data. Fastening takes place while the patient bites into the fitted bite plate in order to be able to undertake a registration of the orientation of the movement recorder in relation to the bite plate. After the registration, the bite plate is removed from the mouth of the patient. A (movement) detector which is fastened at a fixed position to the head of the patient and which registers the movements of the movement recorder from this position serves as a counterpiece to the movement recorder. With the detachably fixed movement recorder and the detachably fixed detector, the patient then carries out a sequence of a plurality of mastication movements, where possible along different trajectories to the extent that this is permitted by the anatomy of their jaw. These mastication movements are recorded by the detector and stored in a third data record as movement data. The movement data represents movement lines of, in each case, individual points that are related to the lower jaw in a three-dimensional coordinate system that has been fixed by the upper jaw.

Finally, the movement data are brought into a common coordinate system (“registered”) with the three-dimensional image data of the first data record so that, on the three-dimensional image data of the upper and lower row of teeth, the movement thereof can be simulated. The three-dimensional image data of the first data record are married to the individual movement data of the patient of the third data record via the registration by way of the second data record.

The mastication movements of the toothed lower jaw in relation to the toothed upper jaw can then be presented on a screen for the purposes of treatment planning. Within the scope of treatment planning, the treating medical practitioner can virtually insert an implant into one of the jaws and observe how the implant fits into the anatomy of the simulated mastication movement.

Via the direct fusion of digital tooth models and movement recordings, the method according to the present invention and the apparatus according to the present invention provide a solution that is suitable in practice and which is at the same time precise. It is thereby possible to present the real individual positions of the rows of teeth of the patient and the movement of the rows of teeth in the 3D volume in a manner that is anatomically precise. It is thereby possible to superimpose the DVT data onto digital models of the upper and lower jaw, which were obtained by intraoral or laboratory-based scanning methods, as a result of which the precondition was created for producing therapeutic edge-to-edge bite aids or tooth replacements with an optimized jaw relation and subjecting the design thereof to the individual movement patterns of the patient.

An advantage of the procedure according to the present invention, which relates to the simulating representation of the mastication movements of upper and lower jaw, is that the patient is spared from radiation exposure by ionizing radiation. The present invention provides that the spatial assignment between the first data record and the third data record is effectuated via the second data record that is recorded on the model and not on the patient.

For the purposes of measuring the movement and registering the movement, use is advantageously made of a system comprising a movement recorder and a detector, the system operating, for example, on an ultrasound time-of-flight basis, i.e., a conversion of times-of-flight of a plurality of acoustic signals into spatial information. A holder (“attachment”) for the movement recorder is detachably fastened to the patient in a para-occlusal manner therefor, the movement recorder in this case being a measurement sensor arc equipped with sound transmitters. The movement recorder has a magnetic coupling via which it can be stably fastened to the lower jaw attachment and to the bite plate. Four ultrasound transmitters are advantageously arranged on the lower jaw in an arcuate manner over a wide area, in front of the mouth and to the sides, via the movement recorder. The detector fastened above consists of three collinearly positioned microphones, which are respectively arranged to the left and to the right, so that, overall, a measurement field which is simultaneously close to the occlusion and close to the joints is defined.

As a counterpart to the measurement sensor arc that moves with the lower jaw, the system comprises a microphone sensor unit affixed to the head, the microphone sensor unit resting against the glabella and the postauricular mastoid on both sides and being affixed by way of a rubber band over the back of the head. A movement measurement is thereby effectuated only via the mastication movement of the lower jaw relative to the head in a very natural fashion during the mastication movements.

For the referencing with the second data record, the bite plate is interposed, the measurement sensor arc is placed on the bite plate, and a measurement is triggered for a few seconds. The bite plate is subsequently removed, the measurement sensor arc is fastened to the attachment, and the actual functional examination is carried out.

The user has all available measured jaw movements and positions after fusioning the second data record and the movement data. On the basis of a selection list, a selection can be made between various jaw bearings. If a movement is selected from the list, a movement track with an anatomical reference is displayed automatically.

The present invention will be described in more detail below on the basis of the drawings.

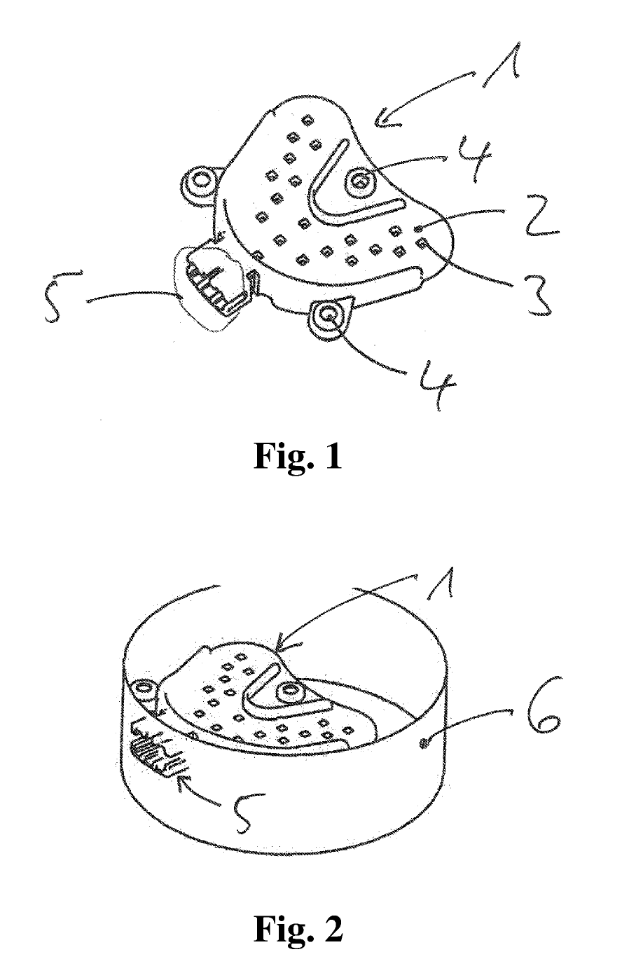

FIG. 1 shows a bite plate 1 which has not yet been equipped with an impression compound at this stage. The impression compound is applied on both sides of the respective bearing face 2 before the patient leaves their bite in the impression compound to be cured. The impression compound is held by teeth 3 which are introduced into the bearing face 2. Markers 4 made of a radiopaque material are attached to the bite plate 1, it being possible to identify the markers 4 in the subsequent x-ray scan. The bite plate 1 also has a plug-in adapter 5 via which the bite plate 1 can be affixed to the recording device. In FIG. 2, the bite plate 1 is surrounded by a cylinder made of a thin sheet 6 which partly absorbs the x-ray radiation during the x-ray scan similar to the tissue of a real patient that surrounds the jaw.

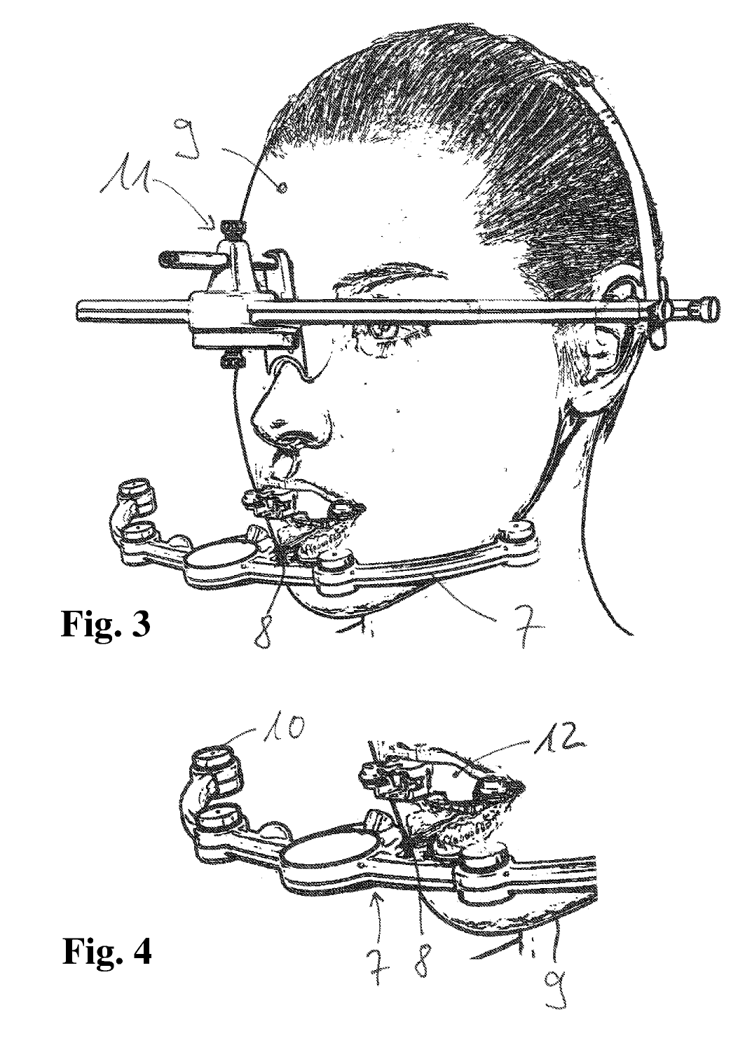

FIG. 3 shows a movement recorder 7 in an arcuate form which, as can be seen from the detailed view according to FIG. 4, is detachably fastened to the lower jaw of a patient 9 by way of a holder 8 (“attachment”). Four ultrasound transmitters 10 are arranged on the movement recorder 7 on both sides. The detector 11 fastened above the forehead of the patient 9 has three collinearly positioned microphones, respectively arranged to the left and to the right, which thereby define the measurement field. In the present case, the patient 9 additionally wears the bite plate 12. In this state, the measurement for registering the movement recorder 7 that has been attached by way of the holder 8 is effectuated, the movement recorder 7 having been fastened, just before this, to the bite plate 12 via an adapter.

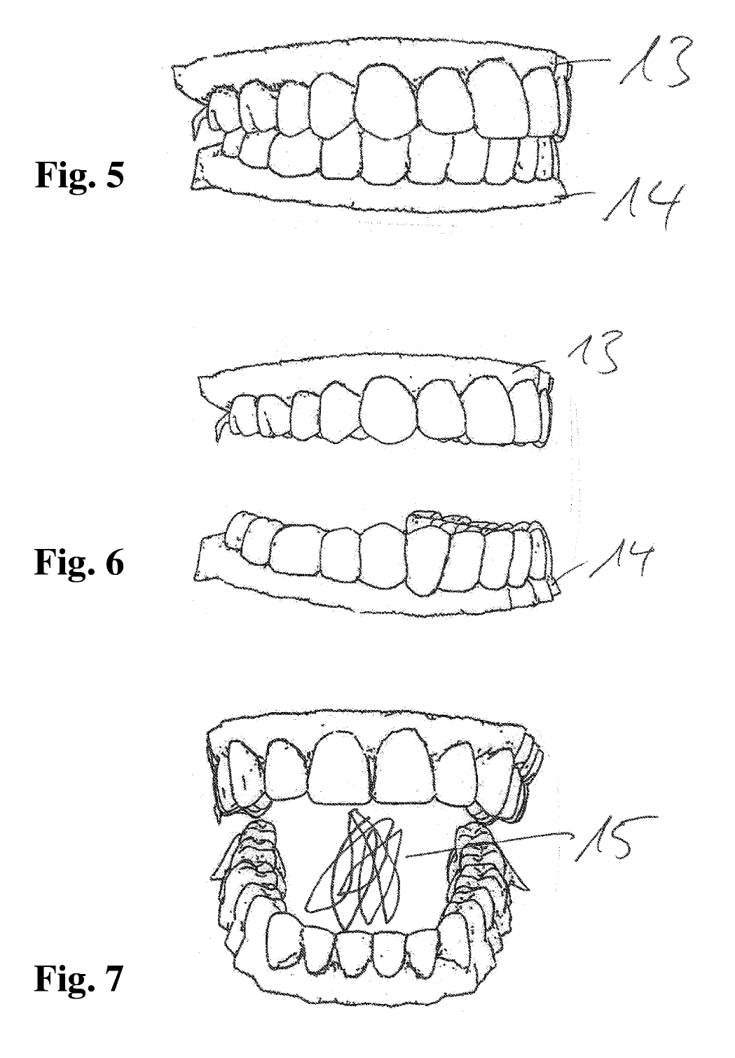

FIGS. 5-7 now show the result of the procedure according to the present invention, namely the rows of teeth of upper jaw 13 and lower jaw 14, presented on a screen, in different simulated movement stages and from different perspectives. In FIG. 5, the set of teeth are completely closed and they are opened to the maximum extent in FIG. 6. FIG. 7 is a frontal view of the open set of teeth. The movement tracks 15 from and during various mastication movements are plotted in the open oral cavity. The treating medical practitioner is now able to plan prosthetics restorations, for example, on the virtual set of teeth.

The individual steps up to this simulation are summarized once again below with the sequence of steps not being restricted to this sequence:
Step 1: recording the digital tooth impressions (first data record);
Step 2: loading the bite plate with the curing impression compound;
Step 3: creating a two-part plaster cast model on the basis of the impression;
Step 4: creating the 3D x-ray scan (second data record) with the bite plate, which is held by the jaws of the plaster cast model;
Step 5: reinserting the bite plate into the mouth of the patient for calibrating the movement-recording device;
Step 6: performing an attachment of the T-attachment to the lower jaw teeth of the patient. The actual movement measurement, after the calibration of the device has been carried out, is effectuated using the T-attachment;
Step 7: performing an initial calibration of the device for the movement measurement with the bite plate;
Step 8: performing a barrier-free movement measurement (third data record) with the movement-recording device without a bite plate;
Step 9: performing a registration of the 3D x-ray data (second data record) and the movement data (third data record) with the aid of the radiopaque spherical markers of the bite plate;
Step 10: performing a registration of the digital tooth impressions (first data record) with the 3D x-ray data (second data record); and
Step 11: performing a movement of the digital tooth impressions (first data record) and of the lower jaw on the basis of the individual movements of the patient (third data record).

According to the present invention, the data of the second data record is only required to indirectly spatially reconcile the data of the first data record and the data of the third data record by way of the data of the second data record.

The present invention is not limited to embodiments described herein; reference should be had to the appended claims.

What is claimed is:

1-10 (canceled)

11. A method for planning a treatment on a set of teeth of a patient, the planning being based on a representation of mastication movements of the patient in which an upper row of teeth and a lower row of teeth move in relation to one another, the method comprising:
recording a first data record with a camera system, the first data record comprising three-dimensional image data of surfaces of an upper jaw which is at least partly toothed and surfaces of a lower jaw which is at least partly toothed;
fitting a bite plate to the patient, the bite plate comprising cured impressions of the upper jaw, cured impressions of the lower jaw, and identifiable markers arranged at least one of at and in the bite plate;
recording a second data record comprising three-dimensional image data which comprises surface data of the bite plate without an involvement of the patient, the second data record allowing for an identification of the surfaces of the upper jaw, the surfaces of the lower jaw, and the identifiable markers of the bite plate therein;
detachably fastening a movement recorder to the lower jaw of the patient while the patient bites on the bite plate fitted on the patient so as to register an orientation of the movement recorder in relation to the bite plate;
removing the bite plate from the patient;
recording movements of the movement recorder during the mastication movements using a detector detachably fastened to a head of the patient and storing the movements in a third data record as movement data;
combining the three-dimensional image data of the first data record with the movement data of the third data record, wherein a registration is performed using the second data record; and
presenting on a screen the mastication movements of the lower jaw in relation to the upper jaw for the purposes of planning the treatment.

12. The method as recited in claim 11, wherein only the bite plate is recorded in the second data record.

13. The method as recited in claim 11, wherein the second data record is recorded based on a model comprising the upper jaw and the lower jaw of the patient and the bite plate, the bite plate being inserted between the upper jaw and lower jaw of the model.

14. The method as recited in claim 13, wherein the model is a plaster cast model.

15. The method as recited in claim 13, wherein the bite plate or the model comprising the bite plate is recorded in the second data record via a three-dimensional x-ray scan.

16. The method as recited in claim 15, wherein the bite plate is surrounded by a metal sheet when recording the second data record via the three-dimensional x-ray scan.

17. The method as recited in claim 16, wherein the metal sheet comprises a varying thickness.

18. The method as recited in claim 11, wherein a holder is used to detachably fasten the movement recorder to the lower jaw of the patient, the holder being affixed to the lower jaw of the patient via an adhesive bonding.

19. The method as claimed in claim 18, wherein,
the movement recorder is initially affixed to the holder of the bite plate in a first orientation, the first orientation of the movement recorder being recorded by the detector,
the movement recorder is then affixed to the holder which is affixed to the lower jaw of the patient via the adhesive bonding in a second orientation, the second orientation of the movement recorder with the bite plate inserted being recorded by the detector, and
the bite plate is then removed to facilitate the mastication movements which are undisturbed.

20. The method as recited in claim 11, wherein,
the three-dimensional image data of the first data record is first combined with the movement data of the third data record so that a registration of the movement data and the data of the second data record initially occurs via the identifiable markers of the bite plate, and
the registration of the three-dimensional image data of the first data record to the data of the second data record subsequently occurs.

21. A system for performing the method as recited in claim 11, the system comprising:
a camera system for recording a first data record comprising three-dimensional image data of surfaces of an upper jaw which is at least partly toothed and surfaces of a lower jaw which is at least partly toothed of a patient;
a bite plate fitted to the patient, the bite plate comprising cured impressions of the upper jaw, cured impressions of the lower jaw, and identifiable markers;
a recording system for creating a second data record of three-dimensional x-ray data comprising surface data of the bite plate for identifying the surfaces of the upper jaw and the surfaces of the lower jaw, and data to identify the identifiable markers;
a movement recorder configured to be detachably fastenable to the lower jaw of the patient;
a device to register an orientation of the movement recorder in relation to the bite plate while the patient bites the bite plate;
a detector detachably fastenable to a head of the patient, the detector being configured to record movements of the movement recorder during mastication movements and to store movement data recorded by the detector in a third data record;
a computer for combining the three-dimensional image data of the first data record with the movement data of the third data record and for registration via the data of the second data record; and
a screen for presenting the mastication movements of the lower jaw in relation to the upper jaw.

22. The system as claimed in claim 21, wherein the bite plate, when fitted to the patient, further comprises a plug-in adapter arranged in a front region of the bite plate, the plug-in adapter being configured to mechanically connect to the recording system for creating the second data record.

23. The system as recited in claim 22, wherein the recording system is an x-ray device.
